(19) United States
(12) Patent Application Publication
(10) Pub. No.: US 2025/0380999 A1
(43) Pub. Date: Dec. 18, 2025

(54) SURGICAL ROBOT

(71) Applicant: LIVSMED INC., Seongnam-si (KR)

(72) Inventors: Se Ra KIM, Suwon-si (KR); So Yeon KIM, Yongin-si (KR); Young Wan KIM, Seongnam-si (KR); Yong Jae SONG, Seongnam-si (KR); Jin Ah SIM, Yongin-si (KR)

(21) Appl. No.: 19/236,508

(22) Filed: Jun. 12, 2025

(30) Foreign Application Priority Data
Jun. 14, 2024 (KR) 10-2024-0077503

(51) Int. Cl. A61B 34/35; A61B 34/30

(52) U.S. Cl. CPC A61B 34/35; A61B 2034/302; A61B 2034/305

(57) ABSTRACT

Provided is a surgical robot to be used in laparoscopic surgery or other various surgeries, including a main body, a first arm unit arranged on one side of the main body and having a first surgical instrument attached thereto, and a second arm unit arranged on another side of the main body and having a second surgical instrument attached thereto.

### CROSS-REFERENCE TO RELATED APPLICATION

This application is based on and claims priority under 35 U.S.C. § 119 to Korean Patent Application No. 10-2024-0077503, filed on Jun. 14, 2024, in the Korean Intellectual Property Office, the disclosure of which is incorporated by reference herein in its entirety.

### BACKGROUND

### 1. Field

The present disclosure relates to a surgical robot, and in particular, to a surgical robot to be used in laparoscopic surgery or other various surgeries.

### 2. Description of the Related Art

Surgery denotes a process of curing illness by cutting, incising, or manipulating the skin, the mucosa layer, and other tissues by using a medical instrument. In particular, laparotomy that treats, shapes, or removes an organ by cutting and opening the skin of a surgical site may cause bleeding, side effects, pain of a patient, scar, etc. Therefore, surgery performed by inserting only a medical instrument, e.g., a laparoscope, a surgical instrument, a microscope for microsurgery, etc. after forming a predetermined hole in the skin, or surgery using a robot has been recently considered as an alternative.

Here, a surgical robot refers to a robot capable of performing surgical action on behalf of a surgeon who has performed the surgical action. Such a surgical robot may perform accurate and precise operations as compared with human beings and may perform a remote surgery.

Surgical robots that are currently being developed worldwide may include bone surgery robots, laparoscopic surgery robots, stereotactic surgery robots, etc. Here, a laparoscopic surgical robot denotes a robot performing a minimal invasive surgery using a laparoscope and surgical tools.

Laparoscopic surgery is a cutting-edge surgical technique in which a laparoscope that is an endoscope for looking inside the abdomen after making a small hole in a navel area and then surgery is performed, and is expected to a large development in the future. Recently, a laparoscope has been provided with a computer chip to obtain more clear and magnified images as compared with the images seen with naked eyes, and has developed so that, when specifically designed laparoscopic surgical instruments are used while watching a screen of a monitor, any kind of surgery may be performed.

Moreover, laparoscopic surgery has advantages such as wide surgery range nearly the same as that of open surgery, less complications than the open surgery, treatment starting soon after the surgery, and high performance of maintaining the physical health or immune function of the patient after the surgery. As such, laparoscopic surgery is gradually recognized as standard surgery for treating colon cancer, etc. in United States, Europe, etc.

In addition, a surgical robot generally includes a master robot and a slave robot. When an operator manipulates a manipulation lever (e.g., a handle) provided on the master robot, an instrument that is coupled to a robot arm of the slave robot or held by the robot arm is manipulated to perform surgery.

The above-mentioned background technology is technical information that the inventor possessed for deriving the present invention or acquired in the process of deriving the present invention, and cannot necessarily be said to be known art disclosed to the general public before filing the application for the present invention.

### SUMMARY

The present disclosure provides a surgical robot for being used in laparoscopic surgery or other various surgeries, the surgical robot including a pair of robot arms on which surgical instruments are attached, wherein a remote center of motion (RCM) point of the surgical instrument may be arranged within an optimal range.

According to an embodiment of the present disclosure, provided is a surgical robot including a main body, a first arm unit arranged on one side of the main body and having a first surgical instrument attached thereto, and a second arm unit arranged on another side of the main body and having a second surgical instrument attached thereto, wherein the first arm unit includes a first arm linear movement portion connected to the main body and linearly movable in a first direction, a first arm connection portion connected to the first arm linear movement portion and configured to determine a remote center of motion (RCM) point of the first surgical instrument, and a first arm extension portion which extends from the first arm connection portion, on which the first surgical instrument is arranged, and which is configured to determine an arrangement angle of the first surgical instrument with respect to the RCM point of the first surgical instrument, the second arm unit includes a second arm linear movement portion connected to the main body and linearly movable in the first direction, a second arm connection portion connected to the second arm linear movement portion and configured to determine an RCM point of the second surgical instrument, and a second arm extension portion which extends from the second arm connection portion, on which the second surgical instrument is arranged, and which is configured to determine an arrangement angle of the second surgical instrument with respect to the RCM point of the second surgical instrument, the first arm connection portion includes a first arm first connection link having one end axially coupled to the first arm linear movement portion, and a first arm second connection link having one end connected to another end of the first arm first connection link and another end to which the first arm extension portion is connected, the second arm connection portion includes a second arm first connection link having one end axially coupled to the second arm linear movement portion, and a second arm second connection link having one end connected to another end of the second arm first connection link and another end to which the second arm extension portion is connected, and at least one of the first arm first connection link, the first arm second connection link, the second arm first connection link, and the second arm second connection link is rotatable about a virtual reference axis so that the RCM point of the first surgical instrument and the RCM point of the second surgical instrument are arranged within a preset range.

In an embodiment of the present disclosure, at least one of the first arm first connection link, the first arm second connection link, the second arm first connection link, and the second arm second connection link may be rotatable about the virtual reference axis so that a distance between a virtual reference point arranged on the main body and the RCM point of one of the first surgical instrument and the second surgical instrument is to be at least ½ of a maximum distance between the RCM point of the other of the first surgical instrument and the second surgical instrument and the reference point.

In an embodiment of the present disclosure, at least one of the first arm first connection link, the first arm second connection link, the second arm first connection link, and the second arm second connection link may be axially coupled so as to be rotatable about the reference axis with a maximum rotation angle of 270° or greater.

In an embodiment of the present disclosure, the first arm connection portion may include a first brake for compensating for a torque generated in one of the first arm first connection link and the first arm second connection link, and the first brake may have a capacity corresponding to at least 1.5 times of a first torque generated in the one of the first arm first connection link and the first arm second connection link due to the rotation of at least one of the first arm first connection link and the first arm second connection link, and corresponding to at most 0.8 times of a second torque that is greater than the first torque and has a preset magnitude.

In an embodiment of the present disclosure, at least one of the first arm first connection link, the first arm second connection link, the second arm first connection link, and the second arm second connection link may be arranged to have an inclination angle ranging from −10° to 10° based on a virtual reference surface that is perpendicular to the reference axis.

In an embodiment of the present disclosure, the first arm unit may include a first bearing arranged on one of the first arm first connection link and the first arm second connection link, and the first bearing may have a static rated load that is at least 1.5 times of a moment caused due to a weight of at least one of the first arm first connection link and the first arm second connection link, and a dynamic rated load that is at least 1.2 times of a moment caused due to a weight of at least one of the first arm first connection link and the first arm second connection link.

In an embodiment of the present disclosure, the main body may include a gravity compensation portion that is connected to each of the first arm unit and the second arm unit for compensating for the gravity applied to the first arm unit and the second arm unit.

In an embodiment of the present disclosure, an inertia moment of the main body may be formed to be at least 1.5 times of a moment applied to the main body when the first arm connection portion and the second arm connection portion are arranged parallel to each other.

In an embodiment of the present disclosure, the gravity compensation portion may include a constant load spring having a capacity greater than or equal to sum of a weight of the first arm unit and a weight of the second arm unit, and the main body may further include a moment compensation portion arranged to compensate for at least some of the moment applied to the main body.

In an embodiment of the present disclosure, the gravity compensation portion may include a weight that is greater than or equal to the sum of the weight of the first arm unit and the weight of the second arm unit, and the moment applied to the main body may be at least partially compensated for by the weight.

In an embodiment of the present disclosure, the first linear movement portion and the second arm linear movement portion may be linearly moved in the first direction so that the RCM point of the first surgical instrument and the RCM point of the second surgical instrument are each arranged between a range from 700 mm to 1300 mm from the ground.

In an embodiment of the present disclosure, the first arm unit further may include a first arm assistant portion that connects the first arm second connection link to the first arm extension portion and adjusts a direction in which the first arm extension portion extends, and the second arm unit may further include a second arm assistant portion that connects the second arm second connection link to the second arm extension portion, and adjusts a direction in which the second arm extension portion extends.

In an embodiment of the present disclosure, the first arm extension portion may include a first arm first extension link having one end axially coupled to the first arm assistant portion, a first arm second extension link having one end axially coupled to another end of the first arm first extension link, and a first arm third extension link which has one end axially coupled to another end of the first arm second extension link and to which the first surgical instrument is connected, the second arm extension portion may include a second arm first extension link having one end axially coupled to the second arm assistant portion, a second arm second extension link having one end axially coupled to another end of the second arm first extension link, and a second arm third extension link which has one end axially coupled to another end of the second arm second extension link and to which the second surgical instrument is connected, at least one of the first arm first extension link, the first arm second extension link, and the first arm third extension link may be rotatable about the RCM point of the first surgical instrument so that the first surgical instrument performs a yaw movement or a pitch movement, and at least one of the second arm first extension link, the second arm second extension link, and the second arm third extension link may be rotatable about the RCM point of the second surgical instrument so that the second surgical instrument performs a yaw movement or a pitch movement.

In an embodiment of the present disclosure, the first arm extension portion may further include a first arm slide link which has one end coupled to another end of the first arm third extension link, on which the first surgical instrument is arranged, and which allows the first surgical instrument to slidably move through driving of a first motor, and the second arm extension portion may further include a second arm slide link which has one end coupled to another end of the second arm third extension link, on which the second surgical instrument is arranged, and which allows the second surgical instrument to slidably move through driving of a second motor.

In an embodiment of the present disclosure, the first arm connection portion may further include a first arm third connection link having one end axially coupled to another end of the first arm first connection link and another end axially coupled to one end of the first arm second connection link, the second arm connection portion may further include a second arm third connection link having one end axially coupled to the other end of the second arm first connection link and another end axially coupled to another end of the second arm second connection link, and at least one of the first arm first connection link, the first arm second connection link, the first arm third connection link, the second arm first connection link, the second arm second connection link, and the second arm third connection link may be rotatable about the reference axis so that the RCM point of the first surgical instrument and the RCM point of the second surgical instrument are arranged within a preset range.

Other aspects, features and advantages other than those described above will become apparent from the following detailed description of the drawings, claims and disclosure.

### BRIEF DESCRIPTION OF THE DRAWINGS

FIG. 1 is a conceptional diagram showing a surgical robot system provided with a surgical robot according to an embodiment of the present disclosure in which:

FIG. 2 is a perspective view of the surgical robot of FIG. 1;

FIG. 3 is a perspective view of a first arm unit of FIG. 2;

FIG. 4 is a perspective view showing an enlarged view of some parts of the first arm unit of FIG. 2;

FIG. 5 is a diagram showing an example arrangement of the surgical robot of FIG. 2;

FIG. 6 is a diagram schematically showing an arrangement range of the surgical instrument of FIG. 2;

FIG. 7 is a diagram showing a gravity compensation portion included in the surgical robot of FIG. 2, according to an embodiment; and

FIG. 8 is a diagram showing a gravity compensation portion included in the surgical robot of FIG. 2, according to another embodiment.

### DETAILED DESCRIPTION

The embodiments will be described below in more detail with reference to the accompanying drawings. Those components that are the same or are in correspondence are rendered the same reference numeral regardless of the figure number, and redundant explanations are omitted.

As the present disclosure allows for various changes and numerous embodiments, particular embodiments will be illustrated in the drawings and described in detail in the written description. The attached drawings for illustrating one or more embodiments are referred to in order to gain a sufficient understanding, the merits thereof, and the objectives accomplished by the implementation. However, the embodiments may have different forms and should not be construed as being limited to the descriptions set forth herein.

In the description, certain detailed explanations of the related art are omitted when it is deemed that they may unnecessarily obscure the essence of the present disclosure.

An expression used in the singular encompasses the expression of the plural, unless it has a clearly different meaning in the context. It will be understood that although the terms “first,” “second,” etc. may be used herein to describe various components, these components should not be limited by these terms. These components are only used to distinguish one component from another. These components are only used to distinguish one component from another.

In the present specification, it is to be understood that the terms such as “including,” “having,” and “comprising” are intended to indicate the existence of the features or components disclosed in the specification, and are not intended to preclude the possibility that one or more other features or components may exist or may be added.

It will be understood that when a unit, region, or component is referred to as being “formed on” another layer, region, or component, it can be directly or indirectly formed on the other layer, region, or component. That is, for example, intervening units, regions, or components may be present.

It will be understood that when an element is referred to as being “connected” or “coupled” to another element, it can be directly connected or coupled to the other element or intervening elements may be present.

Sizes of components in the drawings may be exaggerated for convenience of explanation. In other words, since sizes and thicknesses of components in the drawings are arbitrarily illustrated for convenience of explanation, the following embodiments are not limited thereto.

In the drawings, a plane formed by an X-axis and a Y-axis in a three-axis orthogonal coordinate system may be substantially parallel to the ground on which a surgical robot is arranged, and Z-axis may denote a height direction of the surgical robot. Also, in the description of the disclosure, ‘first direction’ may refer to ‘+Z direction’ or ‘−Z direction’.

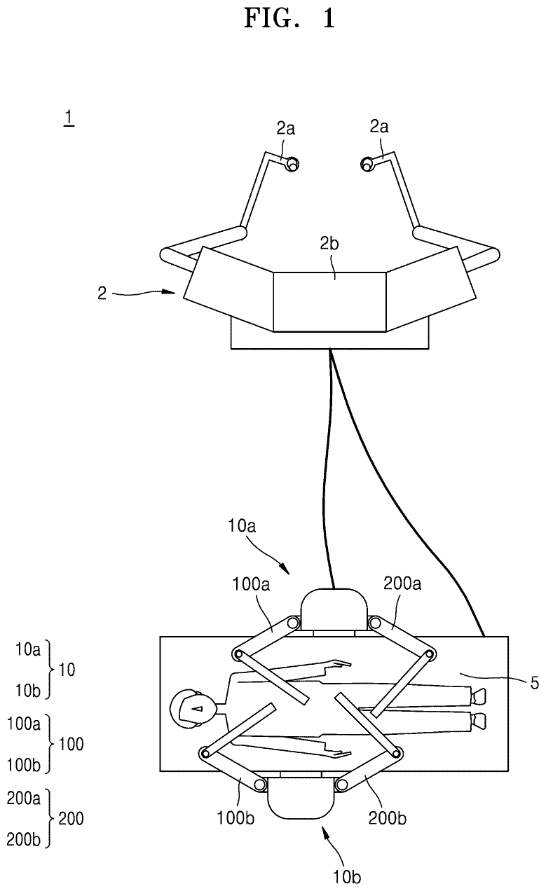

FIG. 1 is a conceptional diagram showing a surgical robot system 1 provided with a surgical robot 10 according to an embodiment of the present disclosure.

Referring to FIG. 1, the surgical robot system 1 may include a master robot 2 and the surgical robot 10.

The master robot 2 includes a manipulation member and a display member, and the surgical robot 10 includes one or more robot arm units 100 and 200.

In detail, the master robot 2 includes manipulation members 2a that may be held and manipulated by both hands of an operator. In addition, images captured by a laparoscope are displayed on a display member 2b of the master robot 2. Also, the display member 2b may display a certain virtual manipulation plate along with the images captured by the laparoscope, etc. or may independently display the virtual manipulation plate. As described above, arrangement, structure, etc. of the virtual manipulation plate are omitted.

In addition, the surgical robot 10 may include at least two robot arm units 100 and 200. Here, each of the robot arm units 100 and 200 may be provided as a module type that may independently operate, and an algorithm for preventing collision between the robot arm units 100 and 200 may be applied to the surgical robot system 1.

The surgical robot system 1 may include one or more surgical robots 10. FIG. 1 shows an example in which the surgical robot system 1 includes two surgical robots 10a and 10b, and each of the surgical robots 10a and 10b includes two robot arm units 100 and 200. Thus, total four robot arm units 100a, 200a, 100b, and 200b are arranged.

In an embodiment, surgical instruments may be applied to two or more of the robot arm units 100 and 200, and a laparoscope may be attached to one or more of the robot arm units 100 and 200. In addition, a surgeon may select one of the robot arm units 100a, 200a, 100b, and 200b that is to be controlled via the master robot 2. As described above, the surgeon may directly manipulate total three or more surgical instruments via the master robot 2, and thus, various tools may be precisely and freely manipulated according to the intention of the surgeon on an operation bed 5 without any surgery assistant.

Hereinafter, detailed configurations and operating principles of the surgical robot 10 are described in detail below.

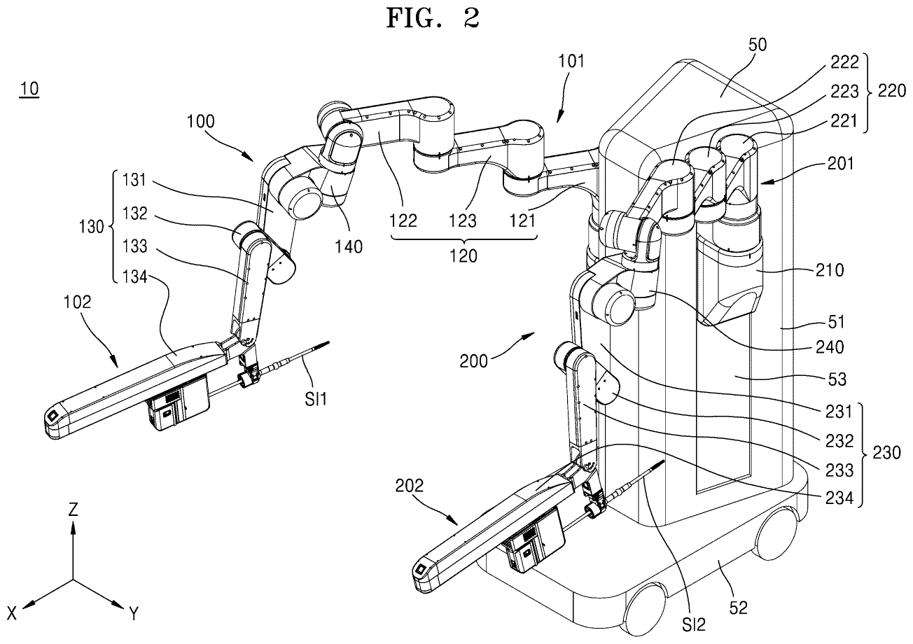

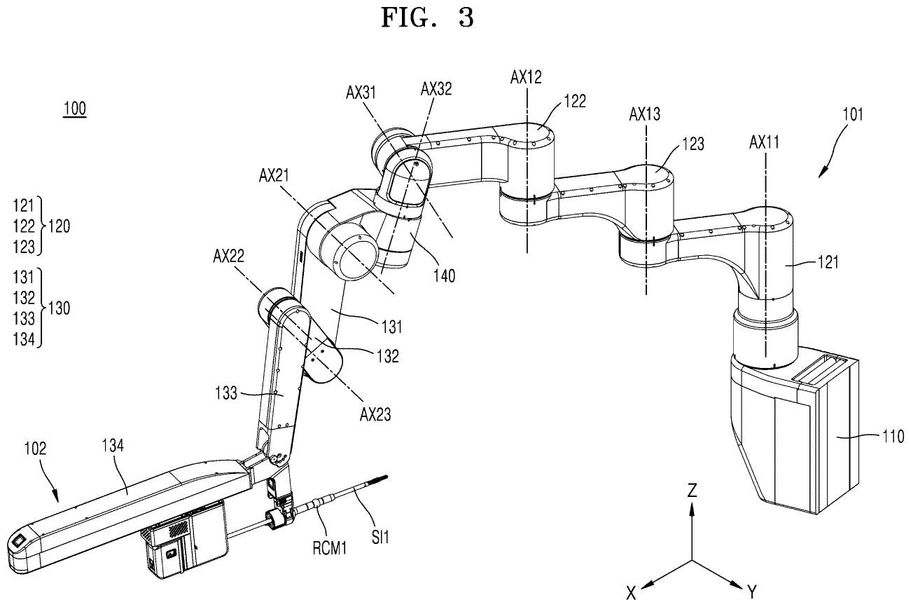

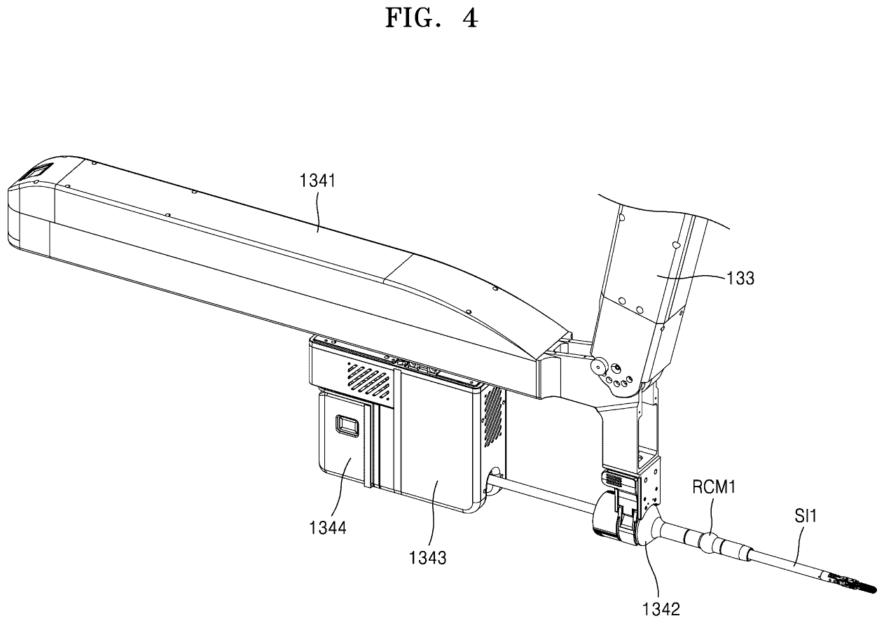

FIG. 2 is a perspective view of the surgical robot 10 of FIG. 1, and FIG. 3 is a perspective view of the first arm unit 100 of FIG. 2. FIG. 4 is a perspective view showing an enlarged view of some parts of the first arm unit 100 of FIG. 2.

Referring to FIGS. 2 to 4, the surgical robot 10 may include a main body 50, the first arm unit 100, and the second arm unit 200. FIG. 2 shows an example in which the surgical robot 10 include two robot arm units, and hereinafter, respective robot arm units are defined as the first arm unit 100 and the second arm unit 200.

The main body 50 acts as a body of the surgical robot 10, and on the main body 50, the first arm unit 100 and the second arm unit 200 may be arranged. Also, the main body 50 may provide a reference point of driving the first arm unit 100 and the second arm unit 200.

The main body 50 may include a first main body 51 and a second main body 52. The first arm unit 100 and the second arm unit 200 are arranged on the first main body 51, and the second main body 52 may support the first main body 51. Also, the second main body 52 may have wheels as shown in FIG. 2, and accordingly, the surgical robot 10 may be moved by means of the wheels.

The main body 50 may have vertical movement guides 53. The number of provided vertical movement guides 53 may correspond to the number of robot arm units arranged on the main body 50. The vertical movement guide 53 may be formed concavely in one side of the main body 50, and respective robot arm units 100 and 200 may be coupled to the vertical movement guides 53 to slidably move in a first direction.

When describing the present disclosure, a part close to the main body 50 is referred to as a proximal end and a part far from the main body 50 is referred to as a distal end. For example, in the first arm unit 100, a part close to the main body 50 is defined as a proximal end 101 of the first arm unit 100, and a part far from the main body 50 is defined as a distal end 102 of the first arm unit 100. Likewise, in the second arm unit 200, a part close to the main body 50 is defined as a proximal end 201 of the second arm unit 200, and a part far from the main body 50 is defined as a distal end 202 of the second arm unit 200.

The first arm unit 100 is arranged on one side of the main body 50 and a first surgical instrument SI1 may be attached to the first arm unit 100. The surgical robot 10 drives the first arm unit 100 to adjust a position and posture of the first surgical instrument SI1.

The first arm unit 100 may include a first arm linear movement portion 110, a first arm connection portion 120, a first arm extension portion 130, and a first arm assistant portion 140.

The first arm linear movement portion 110 is connected to the main body 50 and may linearly move in the first direction. In other words, the first arm linear movement portion 110 is coupled to the vertical movement guide 53 of the main body 50 and may slidably move in the first direction. According to the linear movement of the first arm linear movement portion 110, the first arm connection portion 120 and the first arm extension portion 130 connected to the first arm linear movement portion 110 may be also linearly moved together. That is, the first arm unit 100 is linearly moved in the first direction to adjust a height.

The first arm connection portion 120 may be connected to the first arm linear movement portion 110 to be driven. The first arm connection portion 120 may include a plurality of connection links, and posture of the first arm connection portion 120 may be determined according to the driving of each connection link. A remote center of motion (RCM) point RCM1 of the first surgical instrument SI1 may be determined according to the posture of the first arm connection portion 120. Here, the RCM point of the first surgical instrument SI1 denotes a virtual center point that becomes a base of the rotation of the first surgical instrument SI1. The first surgical instrument SI1 may rotate about the RCM point to perform a yaw movement and a pitch movement.

When each connection link included in the first arm connection portion 120 is driven and the posture of the first arm connection portion 120 is determined, the RCM point RCM1 of the first surgical instrument SI1 is fixed to one point. That is, after the posture of the first arm connection portion 120 is determined by driving each connection link in the first arm connection portion 120, the posture of the first arm connection portion 120 is not changed until the posture is reset. The first surgical instrument SI1 rotates about the RCM point determined according to the posture of the first arm connection portion 120 to perform a yaw movement and a pitch movement.

The first arm extension portion 130 is extended from the first arm connection portion 120, and the first surgical instrument SI1 is attached to the first arm extension portion 130 and an arrangement angle of the first surgical instrument SI1 may be determined.

In detail, the first arm extension portion 130 may have a plurality of extension links and the posture of the first arm extension portion 130 may be determined according to the driving of each extension link. When the posture of the first arm connection portion 120 is determined, the RCM point RCM1 of the first surgical instrument SI1 is fixed, and according to the driving of the first arm extension portion 130, the arrangement angle of the first surgical instrument SI1 may be changed. That is, the first surgical instrument SI1 may rotate about the RCM point RCM1 determined according to the driving of the first arm extension portion 130.

The first arm assistant portion 140 may connect the first arm connection portion 120 and the first arm extension portion 130 to each other. The first arm assistant portion 140 is arranged between the first arm connection portion 120 and the first arm extension portion 130 and may guide the first arm extension portion 130 to have a certain angle based on the first arm connection portion 120. That is, the extension direction of the first arm extension portion 130 may be determined by the first arm assistant portion 140.

The second arm unit 200 is arranged on the other side of the main body 50 and a second surgical instrument SI2 may be attached on the second arm unit 200. The surgical robot 10 drives the second arm unit 200 to adjust a position and posture of the second surgical instrument SI2.

The second arm unit 200 may be arranged on the other side of the main body 50 while opposing the first arm unit 100. For example, the first main body 51 of the main body 50 has a cross-section of roughly trapezoidal shape as shown in FIG. 2, and the first arm linear movement portion 110 of the first arm unit 100 and the second arm linear movement portion 210 of the second arm unit 200 may be arranged to be closer to each other. Alternatively, the first arm linear movement portion 110 of the first arm unit 100 and the linear movement portion of the second arm unit 200 may be parallel to each other on the first main body 51 of the main body 50. According to arrangement angles of the first arm linear movement portion 110 of the first arm unit 100 and the second arm linear movement portion 210 of the second arm unit 200, driving ranges of the first arm unit 100 and the second arm unit 200 may be changed. In other words, according to the arrangement angles of the first arm linear movement portion 110 of the first arm unit 100 and the second arm linear movement portion 210 of the second arm unit 200, a rotating range of each link in the first arm unit 100 and the second arm unit 200 may be formed.

The second arm unit 200 may include the second arm linear movement portion 210, a second arm connection portion 220, a second arm extension portion 230, and a second arm assistant portion 240.

The second arm linear movement portion 210 is connected to the main body 50 and may linearly move in the first direction. In other words, the second arm linear movement portion 210 is coupled to the vertical movement guide 53 of the main body 50 and may slidably move in the first direction. According to the linear movement of the second arm linear movement portion 210, the second arm connection portion 220 connected to the second arm linear movement portion 210 and the second arm extension portion 230 may be also linearly moved together. That is, the second arm unit 200 is linearly moved in the first direction to adjust a height.

The second arm connection portion 220 may be connected to the second arm linear movement portion 210 to be driven. The second arm connection portion 220 may include a plurality of connection links, and posture of the second arm connection portion 220 may be determined according to the driving of each connection link. A remote center of motion (RCM) point RCM2 of the second surgical instrument SI2 may be determined according to the posture of the second arm connection portion 220. Here, the RCM point of the second surgical instrument SI2 denotes a virtual center point that becomes a base of the rotation of the second surgical instrument SI2. The second surgical instrument SI2 may rotate about the RCM point to perform a yaw movement and a pitch movement.

When each connection link included in the second arm connection portion 220 is driven and the posture of the second arm connection portion 220 is determined, the RCM point RCM2 of the second surgical instrument SI2 is fixed to one point. That is, after the posture of the second arm connection portion 220 is determined by driving each connection link in the second arm connection portion 220, the posture of the second arm connection portion 220 is not changed until the posture is reset. The second surgical instrument SI2 rotates about the RCM point RCM2 determined according to the posture of the second arm connection portion 220 to perform a yaw movement and a pitch movement.

The second arm extension portion 230 is extended from the second arm connection portion 220, and the second surgical instrument SI2 is attached to the second arm extension portion 230 and an arrangement angle of the second surgical instrument SI2 may be determined.

In detail, the second arm extension portion 230 may have a plurality of extension links and the posture of the second arm extension portion 230 may be determined according to the driving of each extension link. When the posture of the second arm connection portion 220 is determined, the RCM point RCM2 of the second surgical instrument SI2 is fixed, and according to the driving of the second arm extension portion 230, the arrangement angle of the second surgical instrument SI2 may be changed. That is, the second surgical instrument SI2 may rotate about the fixed RCM point determined according to the driving of the second arm extension portion 230.

The second arm assistant portion 240 may connect the second arm connection portion 220 and the second arm extension portion 230 to each other. The second arm assistant portion 240 is arranged between the second arm connection portion 220 and the second arm extension portion 230 and may guide the second arm extension portion 230 to have a certain angle based on the second arm connection portion 220. That is, the extension direction of the second arm extension portion 230 may be determined by the second arm assistant portion 240.

Next, connection structure and arrangement of respective links included in the robot arm unit of the surgical robot 10 are described in detail based on the first arm unit 100. As described above, the first arm unit 100 may include the first arm linear movement portion 110, the first arm connection portion 120, the first arm assistant portion 140, and the first arm extension portion 130. In particular, the first arm connection portion 120 may include a plurality of first arm connection links and the first arm extension portion 130 may include a plurality of first arm extension links.

First, the first arm connection portion 120 of the surgical robot 10 according to an embodiment of the present disclosure may include a first arm first connection link 121, and a first arm second connection link 122.

One end of the first arm first connection link 121 may be axially coupled to the first arm linear movement portion 110. The first arm first connection link 121 may be axially coupled to the first arm linear movement portion 110 and rotated.

One end of the first arm second connection link 122 is connected to the other end of the first arm first connection link 121 and the other end of the first arm second connection link 122 may be connected to the first arm extension portion 130. Here, the first arm second connection link 122 has one end that is axially coupled directly to the other end of the first arm first connection link 121 and may be rotated.

Alternatively, the first arm connection portion 120 may further include one or more first arm connection links arranged between the first arm first connection link 121 and the first arm second connection link 122, and the first arm second connection link 122 may be connected to the first arm first connection link 121 via the first arm connection links and rotated. That is, the number of first arm connection links in the first arm connection portion 120 is not particularly limited, and the RCM point RCM1 of the first surgical instrument SI1 may be determined according to the arrangement of respective first arm connection links.

Hereinafter, as shown in FIG. 3, an example in which the first arm connection portion 120 includes three first arm connection links and the first arm extension portion 130 includes three first arm extension links is described below. As described above, the first arm connection link coupled to the first arm linear movement portion 110 is defined as the first arm first connection link 121 and the first arm connection link connected to the first arm extension portion 130 is defined as the first arm second connection link 122 in the first arm unit 100, and in FIG. 3, the first arm connection link arranged between the first arm first connection link 121 and the first arm second connection link 122 is defined as a first arm third connection link 123.

Each first arm connection link of the first arm connection portion 120 may be rotated about first reference axes AX10. The first reference axes AX10 that are virtual reference axes that become the centers of rotation may be defined at the proximal end sides of respective first arm connection links. A direction in which the first reference axes AX10 extend may be substantially perpendicular to the lengthwise direction of each first arm connection link. That is, when the first arm connection link is arranged to have a certain angle with respect to an XY plane of the drawing, the first reference axes AX10 may also have a certain angle with respect to the Z-axis of the drawing.

In other words, the direction in which the first reference axes AX10 extend is not necessarily parallel to the Z-axis, but may vary depending on the arrangement of the first arm connection link. The first reference axes AX10 that are the centers of rotation of the first arm first connection link 121, the first arm second connection link 122, and the first arm third connection link 123 are defined respectively as a first-1 reference axis AX11, a first-2 reference axis AX12, and a first-3 reference axis AX13.

One end of the first arm first connection link 121 may be axially coupled to the first arm linear movement portion 110. The first arm first connection link 121 may have one end axially coupled to the first arm linear movement portion 110 so as to be rotatable about one axis formed at the proximal end side. The first-1 reference axis AX11 that is a virtual reference axis connecting one point to the first arm linear movement portion 110 may be formed at the proximal end side of the first arm first connection link 121. The first arm first connection link 121 may be axially coupled to the first arm linear movement portion 110 so as to be rotatable about the first-1 reference axis AX11.

The first arm third connection link 123 may have one end axially coupled to the other end of the first arm first connection link 121. The first arm third connection link 123 may have one end axially coupled to the other end of the first arm first connection link 121 so as to be rotatable about one axis formed at the proximal end side. That is, the first-3 reference axis AX13 that is a virtual reference axis connecting one point to one point at the distal end side of the first arm first connection link 121 may be formed at the proximal end side of the first arm third connection link 123. The first arm third connection link 123 may be axially coupled to the first arm first connection link 121 so as to be rotatable about the first-3 reference axis AX13.

The first arm second connection link 122 may have one end axially coupled to the other end of the first arm third connection link 123. The first arm second connection link 122 may have one end axially coupled to the other end of the first arm third connection link 123 so as to be rotatable about one axis formed at the proximal end side. That is, the first-2 reference axis AX12 that is a virtual reference axis connecting one point to one point at the distal end side of the first arm third connection link 123 may be formed at the proximal end side of the first arm second connection link 122. The first arm second connection link 122 may be axially coupled to the first arm third connection link 123 so as to be rotatable about the first-2 reference axis AX12.

In an embodiment, at least one of the first arm first connection link 121, the first arm second connection link 122, and the first arm third connection link 123 may be axially coupled so as to be rotatable about each reference axis by about a rotation angle of 270° or greater. Because a maximum rotation angle of the first arm connection link is set to be 270° or greater, a movable range of the first arm unit 100 may cover a certain region surrounding the main body 50, and the RCM point may be arranged in the certain region surrounding the main body 50.

The first arm first connection link 121, the first arm second connection link 122, and the first arm third connection link 123 may be each arranged to be inclined based on a virtual first reference surface (not shown). In detail, one of the first arm first connection link 121, the first arm second connection link 122, and the first arm third connection link 123 may be arranged while being tilted with respect to a virtual first reference surface that is perpendicular to each first reference axis AX10. For example, when the first reference axes AX10 are formed parallel to the Z-axis of FIG. 3, the first reference surface (not shown) may be parallel to the XY plane of FIG. 3.

On the contrary, when the first reference axes AX10 are arranged to have a certain angle with reference to the Z-axis of FIG. 3, the first reference surface (not shown) may also have a certain angle with respect to the XY plane of FIG. 3. The virtual first reference surfaces that are the references of arrangement angles of the first arm first connection link 121, the first arm second connection link 122, and the first arm third connection link 123 are respectively defined as a first-1 reference surface, a first-2 reference surface, and a first-3 reference surface.

The first arm first connection link 121, the first arm second connection link 122, and the first arm third connection link 123 may be arranged to be tilted respectively based on the first-1 reference surface, the first-2 reference surface, and the first-3 reference surface. Here, an inclination angle between each of the first arm connection links and each of the first reference surfaces may be restricted to a range of −10° to 10°.

The first arm connection portion 120 may further include a first brake (not shown) and a first bearing (not shown). Once the posture of the first arm connection portion 120 is determined, the posture of the first arm connection portion 120 has to be maintained until the posture is reset. The first arm connection portion 120 includes the first brake (not shown) and the first bearing (not shown) so as to maintain the posture without regard to such factors as the gravity, a torque that is naturally generated, external force, etc.

The first brake (not shown) may compensate for the torque generated in one of the first arm first connection link 121, the first arm second connection link 122, and the first arm third connection link 123. When the posture of the first arm connection portion 120 is determined, the RCM point RCM1 of the first surgical instrument SI1 is fixed, and the first surgical instrument SI1 may perform the surgical operations while rotating about the fixed RCM point.

Therefore, the posture of the first arm connection portion 120 has not to be changed due to the external factors after determining the posture, until the posture is reset. Therefore, the first arm connection portion 120 includes the first brake (not shown) that compensates for the torque generated in the first arm connection links, and thus, even when the torque is generated in the first arm connection links due to the external factors, the torque is offset and the posture of the first arm connection portion 120 may be maintained.

The first brake (not shown) may be arranged on at least one of the first arm first connection link 121, the first arm second connection link 122, and the first arm third connection link 123. That is, the first brake (not shown) may refer to various brake devices arranged on each or some of the first arm first connection link 121, the first arm second connection link 122, and the first arm third connection link 123.

In an embodiment, a capacity of the first brake (not shown) may correspond to at least 1.5 times greater than a first torque generated due to the rotation of at least one of the first arm first connection link 121, the first arm second connection link 122, and the first arm third connection link 123. Each of the first arm connection links of the first arm connection portion 120 may have exclusive first torque due to the rotation, and the first brake (not shown) has the capacity corresponding to at least 1.5 times of the first torque so as to prevent the first arm connection link from naturally rotating during the surgery.

Also, the first brake (not shown) may have a capacity corresponding to at most 0.8 times of a second torque that has a preset size that is greater than the first torque. There may be the case in which the posture of the first arm connection portion 120 has to be changed, e.g., an urgent issue occurs during surgery. In this situation, the external force is applied to the first arm connection portion 120, and the posture of the first arm connection portion 120 may be artificially changed. In addition, when the capacity of the first brake (not shown) is excessively large, the posture of the first arm connection portion 120 may not be changed even when the external force is applied. In the above situation, the first brake (not shown) has to have a capacity less than the torque generated due to the external force so that the posture of the first arm connection portion 120 may be artificially changed.

That is, the first brake (not shown) may have a capacity that is greater than 1.5 times of the first torque generated in the first arm connection link according to the surgical operation and less than or equal to 0.8 times of the second torque having a preset size, and thus, the posture of the first arm connection portion 120 may be artificially changed during the surgery.

For example, the second torque may be set as a torque value generated in one of the first arm first connection link 121, the first arm second connection link 122, and the first arm third connection link 123 due to an average force applied by a general female adult to the distal end side of the first arm connection portion 120. This is an assumption in which a female worker in an operating room applies external force to the first arm connection portion 120, in consideration that a female may generally have less power than a male. Here, an average value of the force that may be applied by a general female adult may be obtained in various methods such as experiments, survey, etc.

The first bearing (not shown) may be arranged on one of the first arm first connection link 121, the first arm second connection link 122, and the first arm third connection link 123. As described above, the first arm first connection link 121, the first arm second connection link 122, and the first arm third connection link 123 may be arranged respectively to have certain inclination angles based on the first reference surfaces. Here, because a moment in a direction sagging to the ground may be applied to the first arm connection link, the first connection portion may include the first bearing (not shown) so as to secure stability in arranging the first arm connection portion 120.

The first bearing (not shown) may include various bearing members having a rated load that is greater than or equal to a moment generated due to the weight of at least one of the first arm first connection link 121, the first arm second connection link 122, and the first arm third connection link 123. For example, when the first bearing (not shown) is arranged at the proximal end side of the first arm first connection link 121, the first bearing (not shown) may have a rated load that is greater than or equal to the moment generated due to the sum of the weights of the first arm first connection link 121, the first arm second connection link 122, and the first arm third connection link 123. Alternatively, when a second bearing is arranged at the proximal end side of the first arm second connection link 122, the first bearing (not shown) may have a rated load that is greater than or equal to the moment generated due to the sum of the weights of the first arm second connection link 122 and the first arm third connection link 123.

In an embodiment, the first bearing (not shown) may have a static rated load that is at least 1.5 times and a dynamic rated load that is at least 1.2 times of the moment that is generated due to the weight of at least one of the first arm first connection link 121, the first arm second connection link 122, and the first arm third connection link 123. The first bearing (not shown) has the sufficient static rated load and dynamic rated load as compared with the weight of the connection links, and thus, the posture of the first arm connection portion 120 may be stably maintained.

As described above, the first arm connection portion 120 may include the first brake (not shown) and the first bearing (not shown) in order to improve the convenience in surgery while stably maintaining the posture. Moreover, although not shown in the drawings, the first arm connection portion 120 may include components such as a motor, a pulley, etc. for rotationally driving each connection link.

The first arm extension portion 130 may include three first arm extension links. The first surgical instrument SI1 may be rotationally moved due to the driving of each first arm extension link. The first arm extension links that are arranged from the proximal end 101 toward the distal end 102 of the first arm unit 100 are defined respectively as a first arm first extension link 131, a first arm second extension link 132, and a first arm third extension link 133.

The respective first arm extension links of the first arm extension portion 130 may rotate about second reference axes AX20. The second reference axes AX20 that are virtual reference axes that become the centers of rotation may be defined at the proximal end sides of respective first arm extension links. A direction in which the second reference axes AX20 extend may be substantially perpendicular to the lengthwise direction of the first arm extension links. That is, the extension direction of the second reference axes AX20 may vary depending on the posture of the first arm connection portion 120 and arrangement of the respective first arm extension links. The second reference axes AX20 that are the centers of rotation of the first arm first extension link 131, the first arm second extension link 132, and the first arm third extension link 133 are defined respectively as a second-1 reference axis AX21, a second-2 reference axis AX22, and a second-3 reference axis AX23.

One end of the first arm first extension link 131 may be axially coupled to the first arm assistant portion 140. The first arm first extension link 131 may have one end axially coupled to the first arm assistant portion 140 so as to be rotatable about one axis formed at the proximal end side. The second-1 reference axis AX21 that is a virtual reference axis connecting one point to one point of the first arm assistant portion 140 may be formed at the proximal end side of the first arm first extension link 131. The first arm first extension link 131 may be axially coupled to the first arm assistant portion 140 so as to be rotatable about the second-1 reference axis AX21.

The first arm second extension link 132 may have one end axially coupled to the other end of the first arm first extension link 131. The first arm second extension link 132 may have one end axially coupled to the other end of the first arm first extension link 131 so as to be rotatable about one axis formed at the proximal end side. The second-2 reference axis AX22 that is a virtual reference axis connecting one point to one point of the first arm second extension link 132 may be formed at the proximal end side of the first arm second extension link 132. The first arm second extension link 132 may be axially coupled to the first arm first extension link 131 so as to be rotatable about the second-2 reference axis AX22.

The first arm third extension link 133 may have one end axially coupled to the first arm second extension link 132. The first arm third extension link 133 may have one end axially coupled to the other end of the first arm second extension link 132 so as to be rotatable about one axis formed at the proximal end side. The second-3 reference axis AX23 that is a virtual reference axis connecting one point to one point of the first arm second extension link 132 may be formed at the proximal end side of the first arm third extension link 133. The first arm third extension link 133 may be axially coupled to the first arm second extension link 132 so as to be rotatable about the second-3 reference axis AX23.

The first arm first extension link 131, the first arm second extension link 132, and the first arm third extension link 133 may respectively rotate about the second-1 reference axis AX21, the second-2 reference axis AX22, and the second-3 reference axis AX23. Also, the first arm first extension link 131, the first arm second extension link 132, and the first arm third extension link 133 may be arranged to be inclined based on virtual second reference surfaces that are perpendicular to the second reference axes. The rotation ranges of the first arm extension links and the inclined arrangements of the first arm extension links refer to the above descriptions about the first arm connection links. Also, a brake and a bearing for compensating for the torque, and components such as a motor, a pulley, etc. for the rotational driving may be arranged on the first arm extension links as well.

The first arm extension portion 130 may further include a first arm slide link 134. The first arm slide link 134 may allow the first surgical instrument SI1 to slidably move.

The first arm slide link 134 may be coupled to the other end of the first arm third extension link 133, that is, the distal end side, and the first surgical instrument SI1 may be arranged on the first arm slide link 134.

The first arm slide link 134 may include a translation arm 1341, an instrument coupling portion 1342, a slide motor pack 1343, and a slide driver 1344.

The translation arm 1341 is coupled to the other end of the first arm third extension link 133 and may be moved along with the first arm third extension link 133. That is, when the first arm third extension link 133 drives, the posture of the translation arm 1341 may be also changed.

The instrument coupling portion 1342 is arranged on the translation arm 1341 and the first surgical instrument SI1 may be coupled to the instrument coupling portion 1342. The first surgical instrument SI1 may be attached through the instrument coupling portion 1342. Also, the first surgical instrument SI1 may be partially supported by the instrument coupling portion 1342 and may be slidably moved.

The RCM point RCM1 of the first surgical instrument SI1 may be formed on one side of the instrument coupling portion 1342. That is, the instrument coupling portion 1342 may provide the RCM point on one side, and the RCM point is a reference point of the rotation, in addition to the yaw movement and the pitch movement of the first surgical instrument SI1. When the posture of the first arm connection portion 120 is determined, the position of the RCM point arranged on the instrument coupling portion 1342 is also determined, and even when the first surgical instrument SI1 slidably moves, the position of the RCM point may be fixed.

The slide motor pack 1343 may provide a driving force for sliding movement of the first surgical instrument SI1. The slide motor pack 1343 may include various components that may generate and transfer electric power, including one or more first motors.

The slide driver 1344 receives the driving power transferred from the slide motor pack 1343 and may allow the first surgical instrument SI1 to slidably move. The first surgical instrument SI1 has one end connected to the slide driver 1344 and may be linearly moved by the slide driver 1344.

The first arm assistant portion 140 is arranged between the first arm connection portion 120 and the first arm extension portion 130 and may connect the first arm connection portion 120 to the first arm extension portion 130.

An end portion at the proximal end side of the first arm assistant portion 140 may be connected to the first arm second connection link 122 and an end portion at the distal end side of the first arm assistant portion 140 may be connected to the first arm first extension link 131. One end of the first arm assistant portion 140 may be axially coupled to the other end of the first arm second connection link 122 so as to be rotatable about one axis formed at the proximal end side. That is, a third-1 reference axis AX31 that is a virtual reference axis connecting one point of the first arm assistant portion 140 to one point at the distal end side of the first arm second connection link 122 may be formed at the proximal end side of the first arm assistant portion 140. The first arm assistant portion 140 may be axially coupled to the first arm second connection link 122 so as to be rotatable about the third-1 reference axis AX31.

Due to the first arm assistant portion 140, the first-2 reference axis AX12 and the second-1 reference axis AX21 may extend in different directions. When the posture of the first arm connection portion 120 and the posture of the first arm assistant portion 140 are determined, the RCM point RCM1 of the first surgical instrument SI1 is fixed and the extension direction of the first arm extension portion 130 may be determined. The first arm extension portion 130 may be driven while each of the extension links is rotated in the determined extension direction.

The first arm assistant portion 140 may rotate about a third-2 reference axis AX32 that extends in the lengthwise direction. That is, the first arm assistant portion 140 may perform a roll rotation about the third-2 reference axis AX32, and accordingly, the first arm extension portion 130 connected to the first arm assistant portion 140 may also rotate about the third-2 reference axis AX32.

The first arm unit 100 may further include one or more first sensors (not shown) for sensing the posture of each link. The first sensors (not shown) may sense a rotation angle or arrangement of each of the first arm connection links 121, 122, and 123, the first arm extension links 131, 132, and 133, the first arm slide link 134, the first arm assistant portion 140, etc. The first arm unit 100 may adjust the posture and arrangement of the first surgical instrument SI1 by driving based on the sensing data obtained from the first sensors (not shown).

Also, the first arm unit 100 may further include one or more second sensors (not shown) that may recognize the kind of the attached first surgical instrument SI1. At least three kinds of surgical instruments may be attached to the first arm unit 100 as the first surgical instrument SI1. The second sensors (not shown) may recognize the kind of the first surgical instrument SI1 attached to the first arm connection portion 120, and the surgical robot 10 may adjust the driving of the first arm unit 100 so that the first surgical instrument SI1 may perform appropriate surgical operations based on recognition data.

The second arm unit 200 may include the second arm linear movement portion 210, the second arm connection portion 220, the second arm assistant portion 240, and the second arm extension portion 230. In particular, the second arm connection portion 220 may include a plurality of second arm connection links and the second arm extension portion 230 may include a plurality of second arm extension links. For example, as shown in FIG. 2, the second arm connection portion 220 may include three second arm connection links and the second arm extension portion 230 may include three second arm extension links.

In the second arm connection portion 220, a second arm first connection link 221, a second arm second connection link 222, and a second arm third connection link 223 may be axially coupled, and in the second arm extension portion 230, a second arm first extension link 231, a second arm second extension link 232, and a second arm third extension link 233 may be axially coupled. In addition, the second arm assistant portion 240 may connect the second arm connection portion 220 to the second arm extension portion 230, and may determine a direction in which the second arm extension portion 230 extends. Also, detailed descriptions about connection structures, arrangements, and detailed structures of respective links included in the second arm unit 200 refer to the above descriptions about the first arm unit 100.

The structures of the first arm unit 100 and the second arm unit 200 included in the surgical robot 10 are described as above. The surgical robot 10 according to the present disclosure includes the pair of robot arm units 100 and 200, each of which may include a plurality of links. Each of the robot arm units 100 and 200 may determine an RCM point of the surgical instruments SI through arrangements and rotations of the links.

Because the RCM point of the surgical instrument SI is a criterion for determining a driving range of the surgical instrument SI, it is very important to adjust the robot arm units 100 and 200 so that the RCM point may be arranged at an optimal position for surgery. Also, when the pair of surgical instruments SI are used in surgery, the pair of robot arm units 100 and 200 have to be adjusted so that the RCM points of respective surgical instruments SI may be arranged within an optimal range along with each other.

That is, in the surgical robot 10 of the present disclosure, at least one of the first arm first connection link 121, the first arm second connection link 122, the first arm third connection link 123, the second arm first connection link 221, the second arm second connection link 222, and the second arm third connection link 223 may be rotated so that the RCM point RCM1 of the first surgical instrument SI1 and the RCM point RCM2 of the second surgical instrument SI2 may be arranged in a preset range. Hereinafter, the range in which the RCM points RCM1 of the first surgical instrument SI1 and the RCM points RCM2 of the second surgical instrument SI2 are arranged according to the driving of each connection link is described in detail below.

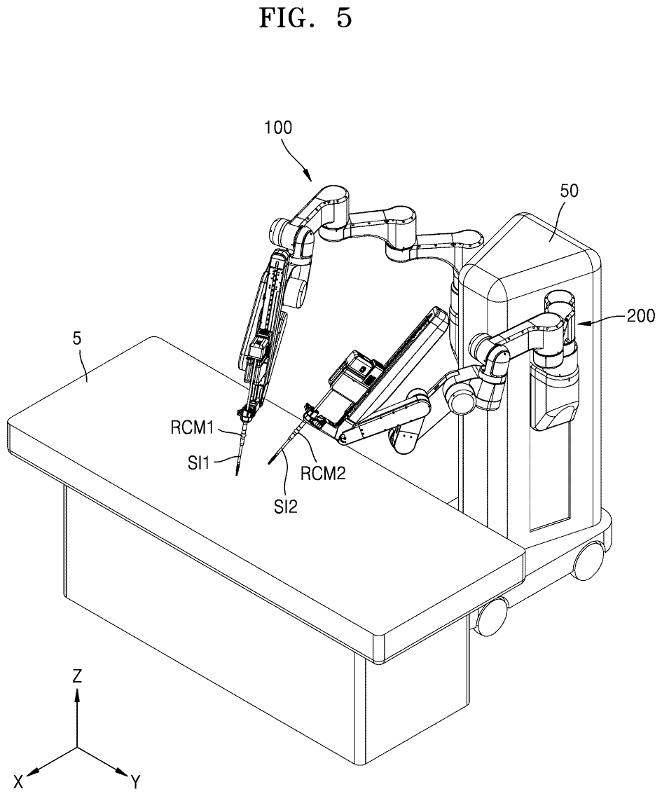

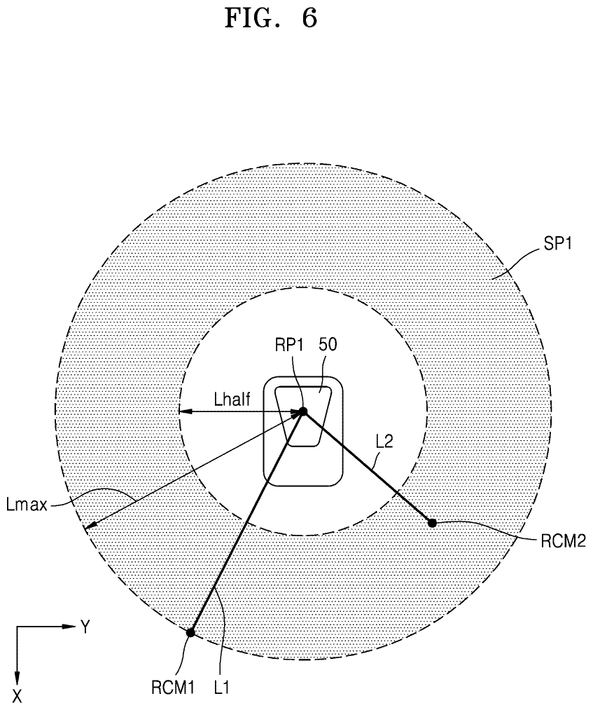

FIG. 5 is a diagram showing an example of the arrangement of the surgical robot 10 of FIG. 1, and FIG. 6 is a diagram schematically showing a movable range of the surgical robot 10 of FIG. 1.

Referring to FIGS. 5 and 6, the RCM point RCM1 of the first surgical instrument SI1 and the RCM point RCM2 of the second surgical instrument SI2 may be arranged in a certain arca including the operation bed 5. The first surgical instrument SI1 and the second surgical instrument SI2 may perform surgical operations through the movements such as a yaw movement, a pitch movement, and a roll movement, etc. on the operation bed 5. Therefore, the RCM points RCM1 of the first surgical instrument SI1 and the RCM points RCM2 of the second surgical instrument SI2 may be arranged on the operation bed 5, or may be arranged at least in the vicinity of the operation bed 5.

In an embodiment, the RCM point RCM1 of the first surgical instrument SI1 and the RCM point RCM2 of the second surgical instrument SI2 may be arranged in a range between 700 mm to 1300 mm from the ground. When the first arm linear movement portion 110 and the second arm linear movement portion 210 of the surgical robot 10 linearly move in the first direction, to adjust the RCM points RCM1 of the first surgical instrument SI1 and the RCM points RCM2 of the second surgical instrument SI2 to be arranged within the above height range. Also, the surgical robot 10 may adjust the height of the RCM point by rotating at least one of the first arm first connection link 121, the first arm second connection link 122, the first arm third connection link 123, the second arm first connection link 221, the second arm second connection link 222, and the second arm third connection link 223. As such, the first surgical instrument SI1 and the second surgical instrument SI2 may be arranged within a certain height range based on the operation bed 5.

First, as described above, the first arm first connection link 121, the first arm second connection link 122, the first arm third connection link 123, the second arm first connection link 221, the second arm second connection link 222, and the second arm third connection link 223 may each rotate about a virtual reference axis. Here, the movable ranges of the robot arm units 100 and 200 may be determined according to the ranges of maximum rotation angles of respective connection links.

For example, at least one of the first arm first connection link 121, the first arm second connection link 122, the first arm third connection link 123, the second arm first connection link 221, the second arm second connection link 222, and the second arm third connection link 223 may be provided to be rotatable with a maximum rotation angle of 270° or greater. As such, the RCM point RCM1 of the first surgical instrument SI1 and the RCM point RCM2 of the second surgical instrument SI2 may be arranged in the entire region surrounding the main body 50.

At least one of the first arm first connection link 121, the first arm second connection link 122, the first arm third connection link 123, the second arm first connection link 221, the second arm second connection link 222, and the second arm third connection link 223 may be rotated so that a distance between the RCM point of one of the first surgical instrument SI1 and the second surgical instrument SI2 and a virtual reference point on the main body 50 may be maintained to be ½ or greater of the maximum distance between another RCM point and the reference point. Here, the virtual reference point may be set on a position corresponding to the center of the main body 50, e.g., one central point of the main body 50 or a center of gravity, etc.

In detail, when respective connection links and the respective extension links included in the first arm unit 100 are all arranged parallel to each other, the distance between the RCM point RCM1 of the first surgical instrument SI1 and the reference point may be the largest. That is, the first arm unit 100 may be arranged and driven within a first circular region having the maximum distance as a radius. Here, the RCM point RCM2 of the second surgical instrument SI2 has to be sufficiently close to the RCM point RCM1 of the first surgical instrument SI1 in order for the first surgical instrument SI1 and the second surgical instrument SI2 to perform the surgery operation along with each other on the operation bed 5.

For example, the RCM point RCM2 of the second surgical instrument SI2 may be arranged in a first effective region SP1 formed between the first circular region having the maximum distance as a radius and a second circular region having half the maximum distance as a radius. Likewise, the RCM point RCM1 of the first surgical instrument SI1 may be arranged in a second effective region formed between a circular region having the maximum distance of the second surgical instrument SI2 as a radius and a circular region having half of the maximum distance as a radius. In other words, the first arm extension portion 130 and the second arm extension portion 230 may be driven so as to maintain the state in which the first surgical instrument SI1 and the second surgical instrument SI2 are arranged in a region greater than or equal to the half of the maximum movable range of each other. As such, the surgical robot 10 may implement optimal arrangement and postures for the surgical instrument SI to effectively perform the surgery operation on the operation bed 5.

The RCM point RCM1 of the first surgical instrument SI1 and the RCM point RCM2 of the second surgical instrument SI2 may be adjusted to be arranged within a certain distance. For example, the RCM point RCM1 of the first surgical instrument SI1 and the RCM point RCM2 of the second surgical instrument SI2 may be adjusted to be arranged within a range having a radius of 15 cm from a surgical site on the operation bed 5. Alternatively, the RCM point RCM1 of the first surgical instrument SI1 and the RCM point RCM2 of the second surgical instrument SI2 may be adjusted to be arranged out of a radius of 200 cm based on the second main body. As described above, because the RCM point RCM1 of the first surgical instrument SI and the RCM point RCM2 of the second surgical instrument SI2 are arranged within a certain region based on the main body 50 and the operation bed 5, the first surgical instrument SI1 and the second surgical instrument SI2 may perform effectively the surgery operations along with each other.

In the above description, the arrangements of the RCM point RCM1 of the first surgical instrument SI1 and the RCM point RCM2 of the second surgical instrument SI2 according to the driving of the surgical robot 10 are described.

In addition, according to the surgical robot 10 of the present disclosure, the pair of robot arm units 100 and 200 having certain weights are coupled to the main body 50, and thus, it is necessary to compensate for the weight of each robot arm unit 100 or 200 for stability. Also, in the surgical robot 10 of the present disclosure, a moment applied to the main body 50 may vary depending on the arrangements and postures of the robot arm units 100 and 200, and accordingly, the surgical robot 10 may collapse to one direction. Hereinafter, a method of compensating for the gravity and compensating for a moment in the surgical robot 10 according to the present disclosure is described below.

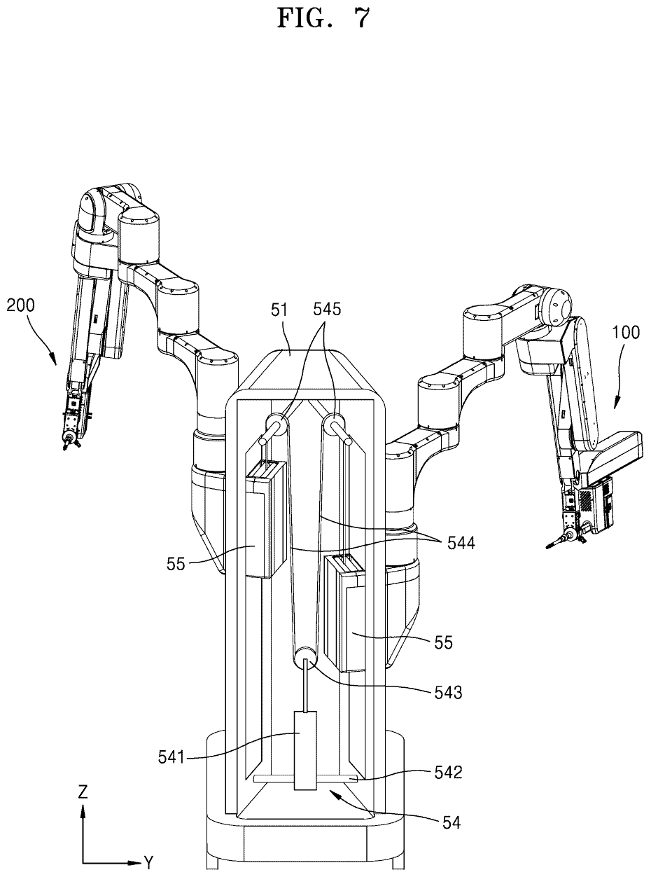

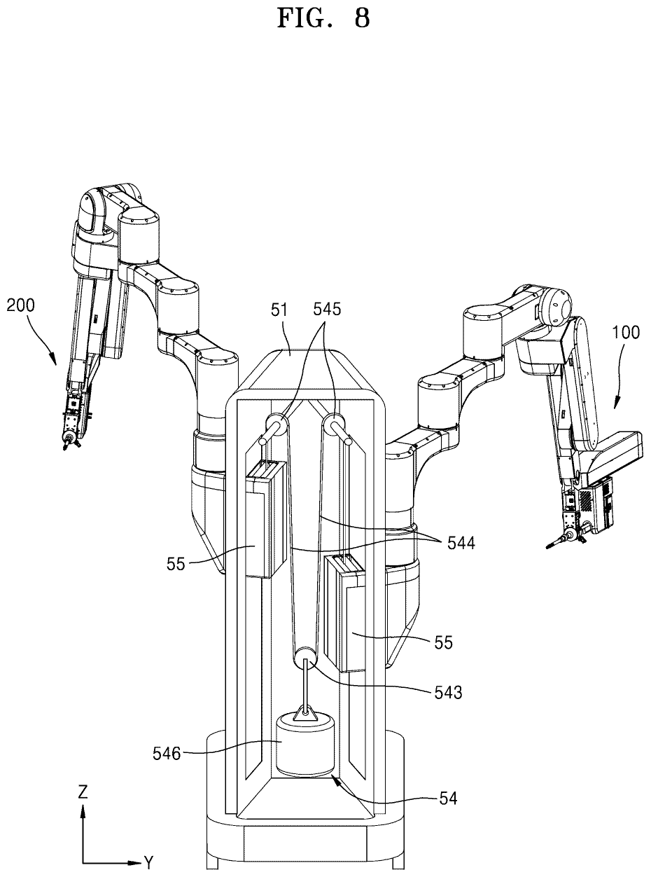

FIG. 7 is a diagram showing an example of a gravity compensation portion 54 in the surgical robot 10 of FIG. 2, and FIG. 8 is a diagram showing another example of the gravity compensation portion 54 of the surgical robot 10.

Referring to FIGS. 7 and 8, the main body 50 of the surgical robot 10 may further include the gravity compensation portion 54. The gravity compensation portion 54 is connected to the pair of robot arm units 100 and 200 coupled to the main body 50, and may prevent the robot arm units 100 and 200 from arbitrarily moving linearly in the first direction after the height of arranging the robot arm units 100 and 200 is determined.

The gravity compensation portion 54 may be connected respectively to the first arm unit 100 and the second arm unit 200, and may include various configurations for offsetting the gravity applied to the first arm unit 100 and the second arm unit 200. FIG. 7 shows an example in which the gravity compensation portion 54 includes a constant load spring 541 for compensating for the gravity of the first arm unit 100 and the second arm unit 200 according to an embodiment, and FIG. 8 shows an example in which the gravity compensation portion 54 includes a weight 546 for compensating for the gravity of the first arm unit 100 and the second arm unit 200 according to an embodiment. Hereinafter, principles of compensating for gravity and compensating for moment in the surgical robot 10 according to the embodiments are described below.

As shown in FIG. 7, the gravity compensation portion 54 may further include the constant load spring 541. The constant load spring 541 is fixed by a fixing shaft 542 in the main body 50 and has one side connected to a moving pulley 543 so as to be extended or contracted according to the movement of the moving pulley 543. Also, the moving pulley 543 may be connected to the first arm unit 100 and the second arm unit 200 via wires 544. Here, the wires 544 are wound around a pair of fixing pulleys 545 and may be arranged to be wound around the moving pulley 543 connected to the constant load spring 541.

The gravity compensation portion 54 may compensate for the gravity by enduring the weights of the first arm unit 100 and the second arm unit 200 via the recovery force of the constant load spring 541. The constant load spring 541 has elastic force and recovery force without regard to the deformation amount, and may have a capacity that is greater than or equal to the sum of the weights of the first arm unit 100 and the second arm unit 200 so as to compensate for the gravity of the first arm unit 100 and the second arm unit 200 together.

In addition, although not shown in FIG. 7, the main body 50 may further include a moment compensation portion (not shown).

When the first arm unit 100 and the second arm unit 200 coupled to the main body 50 are arranged parallel to each other, the total moment applied to the main body 50 may be the maximum due to the weights of the first arm unit 100 and the second arm unit 200. In this case, the surgical robot 10 including the main body 50 may collapse toward a direction in which the first arm unit 100 and the second arm unit 200 are arranged. Therefore, the surgical robot 10 may include an additional moment compensation portion (not shown) in the main body 50 so as to prevent overturn of the main body 50.

In addition, in the embodiment of FIG. 7, the constant load spring 541 compensates for the gravity due to the first arm unit 100 and the second arm unit 200 through an elastic deformation, and thus, the weight of the constant load spring 541 itself may be less than the sum of the weight of the first arm unit 100 and the weight of the second arm unit 200. In this case, because the main body 50 may not have a sufficient weight, the main body 50 may include an additional moment compensation portion (not shown) to compensate for the moment.

For example, the surgical robot 10 may include a component such as an additional weight arranged in the main body 50 as the moment compensation portion (not shown), so that the main body 50 may have a sufficiently heavy weight. Alternatively, the surgical robot 10 may include a moment compensation portion (not shown) that is formed of various components or materials capable of sufficiently lowering the center of gravity of the main body 50. That is, the moment compensation portion (not shown) is provided so that the main body 50 may resist against at least some of the moment generated due to the gravity of the pair of robot arm units 100 and 200, and detailed configurations or types thereof are not particularly limited.

In an embodiment, an inertia moment of the main body 50 may be formed to be at least 1.5 times of the moment applied to the main body 50 when the first arm connection portion 120 and the second arm connection portion 220 are arranged parallel to each other. That is, the moment compensation portion (not shown) may include an additional component such as the weight or appropriately set the material of the main body, and thus, the inertia moment of the main body 50 may be at least 1.5 times of the moment applied to the main body 50 due to the pair of robot arm units 100 and 200. As such, according to the surgical robot 10, the main body 50 and the pair of robot arm units 100 and 200 may be stably arranged.

Next, as shown in FIG. 8, the gravity compensation portion 54 may include the weight 546. The weight 546 may be connected to the moving pulley 543 in the main body 50 and may be moved along with the moving pulley 543. Also, the moving pulley 543 may be connected to the first arm unit 100 and the second arm unit 200 via the wires 544. Here, the wires 544 are wound on a pair of fixing pulleys 545 and may be arranged to be wound around the moving pulley 543 connected to the weight 546.

The gravity compensation portion 54 may compensate for the gravity by enduring the weights of the first arm unit 100 and the second arm unit 200 via the weight 546. The weight 546 may have a weight that is greater than the sum of the weight of the first arm unit 100 and the weight of the second arm unit 200, and may compensate for the gravity with respect to the first arm unit 100 and the second arm unit 200 together.

Unlike the embodiment of FIG. 7, the weight 546 may be greater than or equal to the sum of the weight of the first arm unit 100 and the weight of the second arm unit 200. Therefore, the weight 546 itself may perform as the moment compensation portion (not shown) that compensates for at least part of the moment applied to the main body 50. In addition, the main body 50 may further include components for compensating for another part of the moment applied to the main body 50 so that the main body 50 may have the inertia moment that is at least 1.5 times of the moment applied to the main body 50 due to the pair of robot arm units 100 and 200.

The main body 50 may further include second brakes 55. The second brakes 55 may be connected to the first arm unit 100 and the second arm unit 200 so as to restrict the linear movement of the first arm unit 100 and the second arm unit 200 due to the gravity. That is, the second brakes 55 may prevent the first arm unit 100 and the second arm unit 200 from moving toward the ground due to the gravity. As described above, the surgical robot 10 may prevent the height of arranging each of the robot arm units 100 and 200 from changing without additional adjustment, through the gravity compensation portion 54 and the second brakes 55.

In an embodiment, the second brake 55 may have a capacity corresponding to at least 1.2 times of a magnitude range of the gravity that is not compensated for by the gravity compensation portion 54. For example, when the first arm unit 100 and the second arm unit 200 may each have a weight of 100 N and the gravity compensation portion 54 may compensate for the gravity of 210 N that is greater than the sum of the weights of the two arm units, e.g., 200 N, the brake device may have a capacity of 1.2 times of 10N, that is, the above difference, for example, 12 N or greater. Because the second brake 55 has a sufficiently large capacity in consideration of the gravity compensation amount of the gravity compensation portion 54, the height change of the robot arm units 100 and 200 may be prevented even when the gravities of the robot arm units 100 and 200 are not all compensated for by the gravity compensation portion 54.

The surgical robot according to an embodiment of the present disclosure includes one pair of robot arm units to respectively adjust the first surgical instrument and the second surgical instrument. Here, at least one of the one or more first arm connection links and second arm connection links may be rotated so that the RCM points of the respective surgical instruments may be arranged in a preset region.

The surgical robot according to an embodiment of the present disclosure may compensate for the torque applied to each of the robot arm units provided that an artificial posture change is possible due to the external force. Also, the surgical robot may include the gravity compensation portion and the moment compensation portion and may compensate for the gravities applied to the main body according to the weights of the pair of robot arm units and the moment thereof. As such, the surgical robot may be stably arranged, and unintentional posture change of the robot arm units during the surgery may be prevented.

While the present disclosure has been particularly shown and described with reference to preferred embodiments thereof, it will be understood by those skilled in the art that various changes in form and details may be made therein without departing from the spirit and scope of the present disclosure as defined by the appended claims. The preferred embodiments should be considered in descriptive sense only and not for purposes of limitation. Therefore, the scope of the present disclosure is defined not by the detailed description of the present disclosure but by the appended claims, and all differences within the scope will be construed as being included in the present disclosure.

The surgical robot according to an embodiment of the present disclosure includes one pair of robot arm units, and the first surgical instrument and the second surgical instrument may be respectively attached to the robot arm units. Due to the driving of each robot arm unit, the RCM points of the respective surgical instruments may be arranged along with each other in a preset region so as to effectively perform the surgical operations.

The surgical robot according to an embodiment of the present disclosure may stably maintain the posture of the arm connection portion of each robot arm unit so that the surgical instruments may smoothly perform the surgical operations based on the fixed RCM points. The surgical robot according to an embodiment of the present disclosure may compensate for the torque that is naturally applied to each of the robot arm units, and may artificially change the posture by the external force. Also, the surgical robot according to an embodiment of the present disclosure includes the gravity compensation portion and the moment compensation portion, the heights of the pair of robot arm units are maintained so as not to be changed during the surgery, and the overturn of the main body due to the weights of the robot arm units may be prevented.

What is claimed is:

1. A surgical robot comprising:
a main body;
a first arm unit arranged on one side of the main body and having a first surgical instrument attached thereto; and
a second arm unit arranged on another side of the main body and having a second surgical instrument attached thereto,
wherein the first arm unit comprises:
a first arm linear movement portion connected to the main body and linearly movable in a first direction;
a first arm connection portion connected to the first arm linear movement portion and configured to determine a remote center of motion (RCM) point of the first surgical instrument; and
a first arm extension portion which extends from the first arm connection portion, on which the first surgical instrument is arranged, and which is configured to determine an arrangement angle of the first surgical instrument with respect to the RCM point of the first surgical instrument,
the second arm unit comprises:
a second arm linear movement portion connected to the main body and linearly movable in the first direction;
a second arm connection portion connected to the second arm linear movement portion and configured to determine an RCM point of the second surgical instrument; and
a second arm extension portion which extends from the second arm connection portion, on which the second surgical instrument is arranged, and which is configured to determine an arrangement angle of the second surgical instrument with respect to the RCM point of the second surgical instrument,
the first arm connection portion comprises:
a first arm first connection link having one end axially coupled to the first arm linear movement portion; and
a first arm second connection link having one end connected to another end of the first arm first connection link and another end to which the first arm extension portion is connected,
the second arm connection portion comprises:
a second arm first connection link having one end axially coupled to the second arm linear movement portion; and
a second arm second connection link having one end connected to another end of the second arm first connection link and another end to which the second arm extension portion is connected, and
at least one of the first arm first connection link, the first arm second connection link, the second arm first connection link, and the second arm second connection link is rotatable about a virtual reference axis so that the RCM point of the first surgical instrument and the RCM point of the second surgical instrument are arranged within a preset range.

2. The surgical robot of claim 1, wherein
at least one of the first arm first connection link, the first arm second connection link, the second arm first connection link, and the second arm second connection link is rotatable about the virtual reference axis so that a distance between a virtual reference point arranged on the main body and the RCM point of one of the first surgical instrument and the second surgical instrument is to be at least ½ of a maximum distance between the RCM point of the other of the first surgical instrument and the second surgical instrument and the reference point.

3. The surgical robot of claim 2, wherein
at least one of the first arm first connection link, the first arm second connection link, the second arm first connection link, and the second arm second connection link is axially coupled so as to be rotatable about the reference axis with a maximum rotation angle of 270° or greater.

4. The surgical robot of claim 3, wherein
the first arm connection portion includes
a first brake for compensating for a torque generated in one of the first arm first connection link and the first arm second connection link, and
the first brake has
a capacity corresponding to at least 1.5 times of a first torque generated in the one of the first arm first connection link and the first arm second connection link due to the rotation of at least one of the first arm first connection link and the first arm second connection link, and corresponding to at most 0.8 times of a second torque that is greater than the first torque and has a preset magnitude.

5. The surgical robot of claim 3, wherein
at least one of the first arm first connection link, the first arm second connection link, the second arm first connection link, and the second arm second connection link is arranged to have an inclination angle ranging from −10° to 10° angle based on a virtual reference surface that is perpendicular to the reference axis.

6. The surgical robot of claim 5, wherein
the first arm unit includes
a first bearing arranged on one of the first arm first connection link and the first arm second connection link, and
the first bearing has
a static rated load that is at least 1.5 times of a moment caused due to a weight of at least one of the first arm first connection link and the first arm second connection link, and
a dynamic rated load that is at least 1.2 times of a moment caused due to a weight of at least one of the first arm first connection link and the first arm second connection link.

7. The surgical robot of claim 1, wherein
the main body comprises
a gravity compensation portion that is connected to each of the first arm unit and the second arm unit for compensating for the gravity applied to the first arm unit and the second arm unit.

8. The surgical robot of claim 7, wherein
an inertia moment of the main body is formed to be at least 1.5 times of a moment applied to the main body when the first arm connection portion and the second arm connection portion are arranged parallel to each other.

9. The surgical robot of claim 8, wherein
the gravity compensation portion comprises
a constant load spring having a capacity greater than or equal to sum of a weight of the first arm unit and a weight of the second arm unit, and
the main body further comprises
a moment compensation portion arranged to compensate for at least some of the moment applied to the main body.

10. The surgical robot of claim 8, wherein
the gravity compensation portion comprises
a weight that is greater than or equal to the sum of the weight of the first arm unit and the weight of the second arm unit, and
the moment applied to the main body is at least partially compensated for by the weight.

11. The surgical robot of claim 1, wherein
the first linear movement portion and the second arm linear movement portion
are linearly moved in the first direction so that the RCM point of the first surgical instrument and the RCM point of the second surgical instrument are each arranged between a range from 700 mm to 1300 mm from the ground.

12. The surgical robot of claim 1, wherein
the first arm unit further comprises
a first arm assistant portion that connects the first arm second connection link to the first arm extension portion and adjusts a direction in which the first arm extension portion extends, and
the second arm unit further comprises
a second arm assistant portion that connects the second arm second connection link to the second arm extension portion, and adjusts a direction in which the second arm extension portion extends.

13. The surgical robot of claim 12, wherein
the first arm extension portion comprises:
a first arm first extension link having one end axially coupled to the first arm assistant portion;
a first arm second extension link having one end axially coupled to another end of the first arm first extension link; and
a first arm third extension link which has one end axially coupled to another end of the first arm second extension link and to which the first surgical instrument is connected,
the second arm extension portion comprises:
a second arm first extension link having one end axially coupled to the second arm assistant portion;
a second arm second extension link having one end axially coupled to another end of the second arm first extension link; and
a second arm third extension link which has one end axially coupled to another end of the second arm second extension link and to which the second surgical instrument is connected,
at least one of the first arm first extension link, the first arm second extension link, and the first arm third extension link is rotatable about the RCM point of the first surgical instrument so that the first surgical instrument performs a yaw movement or a pitch movement, and
at least one of the second arm first extension link, the second arm second extension link, and the second arm third extension link is rotatable about the RCM point of the second surgical instrument so that the second surgical instrument performs a yaw movement or a pitch movement.

14. The surgical robot of claim 13, wherein
the first arm extension portion further comprises
a first arm slide link which has one end coupled to another end of the first arm third extension link, on which the first surgical instrument is arranged, and which allows the first surgical instrument to slidably move through driving of a first motor,
and the second arm extension portion further comprises
a second arm slide link which has one end coupled to another end of the second arm third extension link, on which the second surgical instrument is arranged, and which allows the second surgical instrument to slidably move through driving of a second motor.

15. The surgical robot of claim 1, wherein
the first arm connection portion further comprises
a first arm third connection link having one end axially coupled to another end of the first arm first connection link and another end axially coupled to one end of the first arm second connection link,
the second arm connection portion further comprises
a second arm third connection link having one end axially coupled to the other end of the second arm first connection link and another end axially coupled to another end of the second arm second connection link, and
at least one of the first arm first connection link, the first arm second connection link, the first arm third connection link, the second arm first connection link, the second arm second connection link, and the second arm third connection link is rotatable about the reference axis so that the RCM point of the first surgical instrument and the RCM point of the second surgical instrument are arranged within a preset range.
